SEQUENCE LISTING (2 sequences)

SEQ ID NO: 1 - DNA, 20 bp, Artificial Sequence
  PCR primers used to amplify the tomato DET1 genomic DNA fragment flanking the dg mutation locus
ttcttcggattgtccatggt

SEQ ID NO: 2 - DNA, 20 bp, Artificial Sequence
  PCR primer used to amplify the tomato DET1 genomic DNA fragment flanking the dg mutation locus
caccaatgctatgtgccaaa

(19) United States
(12) Patent Application Publication
(10) Pub. No.: US 2008/0184382 A1
(43) Pub. Date: Jul. 31, 2008

(54) High Lycopene Tomato Varieties and Use Thereof

(75) Inventors: Ezri Peleg, Negev (IL); Eyal Vardi, Rehovot (IL)

Correspondence Address: FENNEMORE CRAIG, 3003 NORTH CENTRAL AVENUE, SUITE 2600, PHOENIX, AZ, 85012, US

(73) Assignee: Hazera Genetics LTD.

(21) Appl. No.: 10/587,789

(22) PCT Filed: Jan. 29, 2004
(86) PCT No.: PCT/IL2004/000087

§ 371 Date: Mar. 3, 2008

(51) Int. Cl. A01H 5/00; A01H 1/02

(52) U.S. Cl. 800/260; 800/317.4; 800/300; 800/301; 800/302; 800/303

(57) ABSTRACT

The present invention relates to hardy tomato (Lycopersicon esculentum) varieties, homozygous for the dark green (dg) gene, producing fruit comprising an average lycopene content at least two fold its content in fruit of currently available commercial tomato varieties, wherein the varieties are adapted for growth on a commercial scale and the fruit crop maintains the average high lycopene content. The present invention further relates to the use of said varieties for the production of tomatoes for the fresh and processed fruit markets as well as for the production of lycopene and products comprising same.

### FIELD OF THE INVENTION

The present invention relates to hardy tomato (Lycopersicon esculentum) varieties, homozygous for the dark green (dg) gene, producing fruit comprising an average lycopene content at least two fold its content in fruit of currently available commercial tomato varieties, wherein the varieties are adapted for growth on a commercial scale and the fruit crop maintains the average high lycopene content. The present invention further relates to the use of said varieties for the production of tomatoes for the fresh and processed fruit markets as well as for the production of lycopene and products comprising same.

### BACKGROUND OF THE INVENTION

Plants respond to light intensity, direction, duration, and spectral quality by modulating their developmental processes in an array of interactions that are referred to as photomorphogenesis. Photomorphogenic mutants have been proven to be an excellent tool in the research of the complex interactions between light and plant development and some of them have also been used in several agricultural crop-breeding programs. Photomorphogenic mutants have been reported in a number of species, including Arabidopsis, Sorghum, Brassica, tobacco, tomato and pea. In general, these mutants may be classified either as defective in photoreceptors, or altered in some aspect of the light signal transduction chain (Chory, J. 1993. Trends Genet 9:167-172).

Several light-hypersensitive mutants have been described in tomato (Lycopersicon esculentum). Among these, mutants carrying the monogenic, recessive high pigment (hp-1 and hp-2) and dark green (dg) mutations are characterized by a very high responsiveness to light. A multi-generation allele test suggests that the tomato mutations dg and hp-2 are allelic (Levin et al. Theor. Appl. Genet. 106, 454-460, 2003), and further describes the inferior growth characteristics linked to these mutations in unselected populations. WO 03/057917 discloses use of a genetic marker for detecting the presence of the dg mutation in a plant.

International Patent Application WO 99/29866 discloses the cloning and sequencing of the HP-2 gene. The HP-2 gene was found to encode a tomato homolog to the Arabidopsis nuclear protein DEETIOLATED1 (DET1). The hp-2 mutation is located within the second putative nuclear localization signal of DET1.

The dg tomato mutant is phenotypically similar to other hp mutants, but has a much darker mature green fruit, resulting from higher chlorophyll content. Levin et al., (Theor Appl Genet 106:454-460, 2003) showed that the dg mutation is located on tomato chromosome 1, and is an alternative allele at the HP-2 locus, identified as the tomato DET1 gene. In contrast to the hp-2 mutations, located at the C-terminus of the tomato DET1 protein, the dg mutation was located at the N-terminus of the protein, suggesting that both ends of the protein are important for its function.

The hp and dg mutants display higher anthocyanin levels, shorter hypocotyls, and greater fruit pigmentation in comparison to wild type plants with corresponding genetic background apart from the dg mutation (Wann, et al., J Am Soc Hort Sci 110:212-125, 1985). The increased fruit pigmentation seen in these mutants is due to significantly elevated levels of flavonoids and carotenoids, primarily lycopene, in the mature ripe red fruit.

Lycopene is a potent antioxidant and free radical quencher. It is a natural carotenoid that gives the red color to many fruits, and is found in high amounts in tomatoes and in tomato-derived products. Lycopene has been found to be concentrated in various body tissues, such as liver, adrenal, and adipose tissues, as well as in the prostate. In vitro studies have shown that lycopene has a growth inhibitory effect on mammary, lung, and endometrial carcinoma cell proliferation at and an inhibitory effect on prostate carcinoma cell proliferation at concentrations of 50 μM and above. Although little physiological significance can be attributed to this finding as the inhibitory concentrations in vitro far exceeded typical physiological plasma concentrations for lycopene, epidemiological studies have associated lycopene intake with a decreased risk of various types of cancer, specifically prostate cancer.

U.S. Pat. No. 5,827,900 discloses that lycopene is effective in reducing the overall activity of a cell, and provides a method for inhibiting the growth of cancerous cells by administering an effective cell activity-reducing amount of lycopene.

U.S. Pat. No. 6,555,134 describes a synergistic pharmaceutical or dietary composition containing lycopene and garlic, disclosing that lycopene is highly efficient in quenching singlet oxygen and has a protective effect against oxidative modification of LDL, thus effective in the prevention or treatment of atherosclerosis.

U.S. Pat. No. 6,482,447 shows that compositions comprising lycopene among other plant extracts are useful for treating various conditions and diseases, including benign prostatic hypertrophy (BPH) and prostate cancer. The anti oxidative activity of lycopene reduces free oxygen radicals and therefore reduces BPH, to prevent prostate cancer.

Tomato crops having the hp and dg mutants have a direct application in the agricultural industry as fresh and supplementary food products rich in lycopene are highly desirable. Plants carrying the dg mutation are advantageous over plants carrying an hp mutation, as they produce higher quantities of lycopene. However, hitherto, such varieties were not available in a commercial scale due to pleiotropic, undesired traits linked to the dg mutation (Sacks E. K. and Francis, D. M. 2001. J Amer Hort Sci 126(2):221-226).

Thus, there is a great need for, and it would be highly advantageous to have tomato varieties, homozygous for the dg mutation thus producing fruit with high amounts of lycopene, devoid of the undesired pleiotropic traits associated with this mutation in unselected varieties.

### SUMMARY OF THE INVENTION

The present invention relates to hardy tomato varieties, producing high lycopene fruit for the fresh fruit market as well as for the tomato processing industry. Specifically, the present invention relates to hardy tomato varieties homozygous for the dg mutation, producing fruit comprising an average lycopene content of at least two fold its content in currently available commercial varieties, while devoid of undesirable traits hitherto linked to the dg mutation. The present invention further relates to seeds of the varieties of the present invention, to plants grown from the seeds, to their progeny, to fruit produced by the plants, to plant parts derived therefrom and to methods of producing these varieties. The present invention also relates to products obtained from the high lycopene tomato fruit produced by the varieties of the present invention.

According to one aspect, the present invention provides high lycopene tomato varieties for fresh produce as well as for the industrial market.

According to one embodiment, the present invention provides tomato seeds homozygous for the dg mutation, wherein the plants grown from the seeds yield fruit crops comprising an average lycopene content of at least two fold its content in currently available crop yields, while devoid of deleterious traits associated with the dg mutation.

As used herein in the specification and in the claims section that follows, the deleterious traits associated with the dg mutation, defined also as pleiotropic traits, include, inter alia, poor germination rate; shallow root system; brittle stems; thin and/or fragile leaves; premature defoliation; low yield; small fruit.

According to one embodiment the plants grown from the tomato seeds of the present invention are stable parent plant lines.

According to another embodiment, the plants grown from the tomato seeds of the present invention are F₁ hybrid plant varieties. Within the scope of the present invention the term hybrid varieties encompasses any robust hybrid variety that is homozygous for the dg mutation devoid of the traits poor germination rate, shallow root system, brittle stems, thin and/or fragile leaves, premature defoliation, low yield and small fruit. The hybrid varieties advantageously can further comprise beneficial agronomical traits as are well known in the art including but not limited to disease resistance and various types of stress resistance. Representative hybrid seeds and plant varieties according to the present invention include the varieties designated HA3512, HA3513, HA3518 and HA3519. The F₁ hybrid varieties of the present invention are superior over the parent lines in their plant vigor and adaptation for growth in a commercial scale, including field resistance to various diseases and better yield. The varieties of the invention are preferably non-GMO however it is to be understood that the addition or deletion of traits by transformation is explicitly encompassed within the scope of the invention.

According to one currently preferred embodiment, the present invention provides tomato hybrid seeds designated HA3518. Hybrid HA3518, representative seeds of which have been deposited with the American Type Culture Collection Association on Jan. 29, 2004 (Accession No. not available), serves as an example for the varieties of the present invention, wherein the plants grown from the seeds are homozygous for the dg mutation, produce fruit crop yield comprising an average lycopene content at least two fold its content in currently available crop yields, and devoid of the dg linked deleterious effects.

According to another embodiment, the present invention provides tomato plants and parts thereof producing fruit crop yield comprising an average lycopene content of at least two fold its content in currently available crop yields, wherein the plants are homozygous for the dg mutation while devoid of the dg linked deleterious traits.

According to one embodiment, the present invention provides tomato plants grown from the seeds of hybrid varieties HA3512, HA3513, HA3518 and HA3519.

Pollen and ovules from these tomato plants; the seeds produced from same and the plants grown from the seeds; plants regenerated form tissue cultures regenerated from the plants of the present invention; and plants or parts thereof having all of the physiological and morphological characteristics of the tomato plants of the present invention are also encompassed within the scope of the present invention.

According to one embodiment, the present invention provides a tissue culture regenerated from the tomato plants of the present invention, wherein the tissue culture comprises cells or protoplasts from a tissue selected from the group consisting of leaves, pollen, embryos, roots, root tips, anthers, flowers, fruit and seeds.

According to one embodiment, the average lycopene content in a crop yield is at least 200 ppm. It is to be understood that these values vary greatly depending on the stage of ripening, the conditions of cultivation, the measuring methods used and additional factors. The value of 200 ppm is an average value obtained by measuring the average content of the crop at its peak lycopene production, namely consisting of the ripe red tomatoes obtained by stress free cultivation. Hitherto, such high lycopene content was only found sporadically in fruit of a single plant. The novel varieties of the present invention including parental lines or hybrids adapted for commercial cultivation produce commercial scale crop yields, in which the average lycopene content is at least 200 ppm.

According to another embodiment, the hybrid varieties of the present invention are devoid of the deleterious traits associated with the dg mutation, including, inter alia, poor germination rate; shallow root system; brittle stems; thin and/or fragile leaves; premature defoliation; low yield; small fruit.

According to another aspect, the present invention provides high lycopene tomato fruit. The fruit can be marketed as a fresh product or can serve as a source for processed high lycopene tomato products and purified lycopene.

According to one embodiment, the average lycopene content of the fruits is at least two fold compared to its content in other commercially available fruits.

According to one currently preferred embodiment, the average lycopene content of the fruits is at least 200 ppm plus or minus the standard error from the mean.

According to another aspect, the present invention provides a method for producing first generation (F₁) hybrid tomato seeds.

According to one embodiment, the present invention provides a method for producing first generation hybrid seeds comprising crossing a first parent tomato plant with a second parent tomato plant and harvesting the resultant hybrid F₁ seeds, wherein the first and the second parent plants are homozygous for the dg mutation while devoid of the deleterious effects linked to the dg mutation.

According to another embodiment, the present invention also provides a first generation F₁ hybrid tomato plants that are produced by growing the hybrid tomato seeds produced by the above-described method and to seeds harvested on this hybrid tomato plants and plants grown from these seeds. Tomato plants having within their pedigree a tomato variety homozygous for the dg mutation producing fruit crop yield which comprises an average lycopene content of at least two fold its content in currently available crop yields, while being devoid of the deleterious traits associated with the dg mutation according to the present invention are also included within the scope of the present invention.

According to one embodiment, the present invention encompasses tomato plants having within their pedigree a tomato variety selected from the group consisting of hybrid HA3512, hybrid HA3513 hybrid HA3518 and hybrid HA3519. Hybrid HA3518 serves as an example for the teaching of the present invention, and representatives of its seeds have been deposited with the ATCC on Jan. 29, 2004.

According to yet another aspect, the present invention provides a method for producing tomato plants using the varieties of the present invention, including progeny of the F₁ through F₇ breeding lines and backcrosses thereof.

According to one embodiment, the present invention provides a method of producing a tomato plant derived from a hybrid tomato variety according to the present invention, comprising:
a) crossing a first plant that is a hybrid plant homozygous for the dg mutation according to the present invention with a second tomato plant to yield first progeny seeds;
b) growing the first progeny seed under suitable plant growth conditions to yield an F₁ tomato plant of the first hybrid plant; optionally
c) crossing the plant obtained in step (b) with itself or with a third tomato plant to yield second progeny seeds derived from said first hybrid plant;
d) growing the second progeny seed under suitable plant growth conditions to yield additional tomato plant derived of said first hybrid plant; and further optionally
e) repeating the steps of crossing and growing from 1 to 5 or more times to generate further tomato plants derived from said first hybrid plant.

According to one embodiment, the hybrid tomato variety used as a first plant in the method described above is selected from the group consisting of hybrid HA3512, HA3513, HA3518 and HA3519; these hybrids generally are equivalent to hybrid HA3518, representative seeds of which have been deposited with the ATCC on Jan. 29, 2004 (Accession number not available).

According to another embodiment, the present invention provides plants derived from a dg homozygous plant according to the present invention produced by the method described above.

According to yet another embodiment, the present invention provides robust hybrid tomato plants homozygous for the dg mutation according to the present invention, wherein the plants or progeny or parts thereof have been transformed so that its genetic material contains one or more transgenes operably linked to one or more regulatory elements. Tomato plants and parts thereof produced from the transformed hybrid plants are also encompassed within the scope of the present invention. According to on embodiment, the transformed gene or genes confer a characteristic selected from the group consisting of herbicide resistance, insect resistance, resistance to bacterial, fungal or viral disease, male sterility and improved nutritional value.

According to another aspect the present invention provides a method for selecting a robust tomato variety homozygous for the dg mutation, wherein tomatoes grown from this variety have an average lycopene content at least two fold its average content in currently available varieties, while being devoid of deleterious traits associated with the dg mutation, when measured at peak lycopene content, comprising determining the presence of the dg mutation by using a DNA probe specific for the dg mutation. Suitable probes include but are not limited to those disclosed in WO 03/057917.

The present invention is explained in greater detail in the description, Figures and claims below.

### BRIEF DESCRIPTION OF THE FIGURES

FIG. 1 describes tomato plant homozygous for the dg mutation, which retaining (A) or devoid of (B) the pleiotropic effects associated with the mutation.

FIG. 2 shows a comparison of average fruit yield obtained for hybrid HA3518 and other commercially available hybrids.

### DETAILED DESCRIPTION OF THE INVENTION

The present invention relates to the ongoing need for superior commercial cultivars to meet with market requirements. Such requirements cover a wide area of interest including cultivars with better agronomic traits, better crop yield, improved nutritional value, improved appearance and more.

Particularly, the present invention relates to tomato cultivars producing high lycopene fruit. Such fruits are highly desirable in the fresh tomato market as well as in the tomato processing industry and as a source for purified lycopene.

The present invention provides tomato varieties which are homozygous for the dark green (dg) mutation. As described herein above, tomato plants carrying the dg mutation are characterized by their dark-green fruits, which, upon ripening, become dark red, due to a high lycopene content. Tomato plants carrying the dg mutation, and even plants homozygous for the dg mutation were described before; however, the pleiotropic affects of the this mutation, which include undesirable agronomical traits, prevented the use of the high pigment dg gene in breeding programs. The present invention now discloses novel, hardy hybrid tomato varieties which are dg homozygous, comprising a very high lycopene content while being devoid of the undesired pleiotropic effects associated with the dg mutation.

The development of a commercial, superior tomato variety requires a significant breeding effort, especially what was required to break the linkage between the dg locus and the associated deleterious genes. This association was previously described as a pleiotropic effect, due to the difficulty previous researchers had in breaking this linkage The method chosen for breeding or selection depends on the mode of plant reproduction, the heritability of the trait(s) being improved, and the cultivar (i.e. variety) to be developed commercially (e.g. F₁ hybrid, or an open-pollinated variety). The complexity of the inheritance influences the choice of breeding method. One simple method of identifying a superior plant is to observe its performance relative to other experimental plants or to a widely grown standard cultivar, and to observe its performance in hybrid combinations with other plants. If single observations are inconclusive for establishing distinctness, observations in multiple locations and seasons provide a better estimate of its genetic worth. Proper testing and evaluation should detect any major faults and establish the level of superiority or improvement over current cultivars.

The development of commercial tomato hybrids requires the development of homozygous stable parental lines. In breeding programs desirable traits from two or more germplasm sources or gene pools are combined to develop superior breeding varieties. Desirable inbred or parent lines are developed by continuous selfing and selection of the best breeding lines, sometimes utilizing molecular markers to speed up the selection process.

Once the parent lines that give the best hybrid performance have been identified, the hybrid seed can be produced indefinitely, as long as the homogeneity and the homozygosity of the parents is maintained. A single-cross hybrid is produced when two parent lines are crossed to produce the F₁ progeny. Much of the hybrid vigor exhibited by F₁ hybrids is lost in the next generation (F₂). Consequently, seed harvested from hybrid varieties is not used for planting stock.

According to one embodiment, the present invention provides tomato seeds homozygous for the dg mutation, wherein the plants grown from the seeds yield fruit crops comprising an average lycopene content at least two fold its content in currently available crop yields, while devoid of deleterious traits associated with the dg mutation. The deleterious traits include, inter alia, poor germination rate; shallow root system; brittle stems; thin and/or fragile leaves; premature defoliation; low yield; small fruit.

According to one embodiment the plants grown from the tomato seeds of the present invention are stable parent plant lines.

According to another embodiment, the plants grown from the tomato seeds of the present invention are F₁ hybrid varieties.

As defined herein, stable parent lines refers to open pollinated, inbred lines, stable for the desired plants over cycles of self-pollination and planting. The stable parent lines of the present invention were developed from a cross between plants carrying the dg mutation (the dg mutant of L. esculentum cv Manapal) and a mixture of germplasm from proprietary and valuable breeding material belonging to Hazera Genetics Ltd., the applicant of the present invention. The selection method included the following steps:

Step 1: F₁ populations resulted from the above described crosses were self-crossed to obtain various F₂ populations, in which, theoretically, one forth is homozygous for the dg mutation.

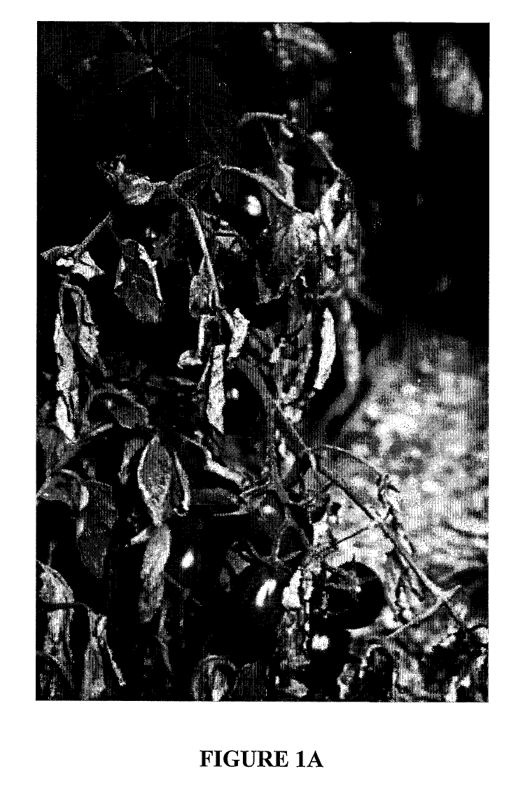

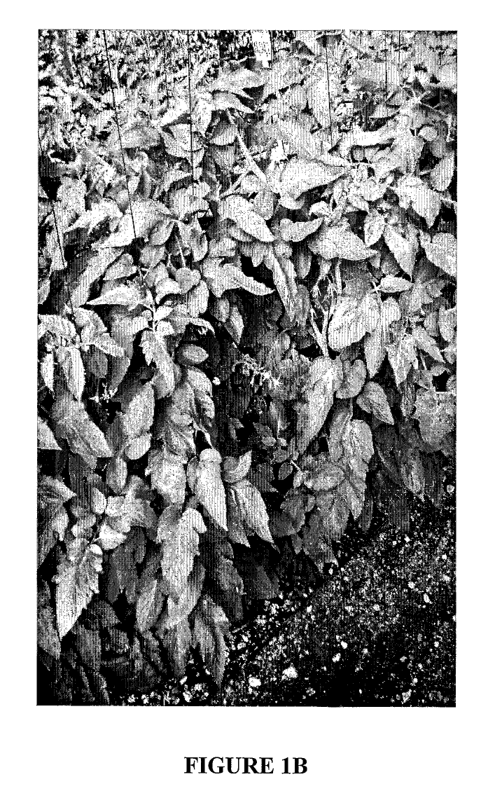

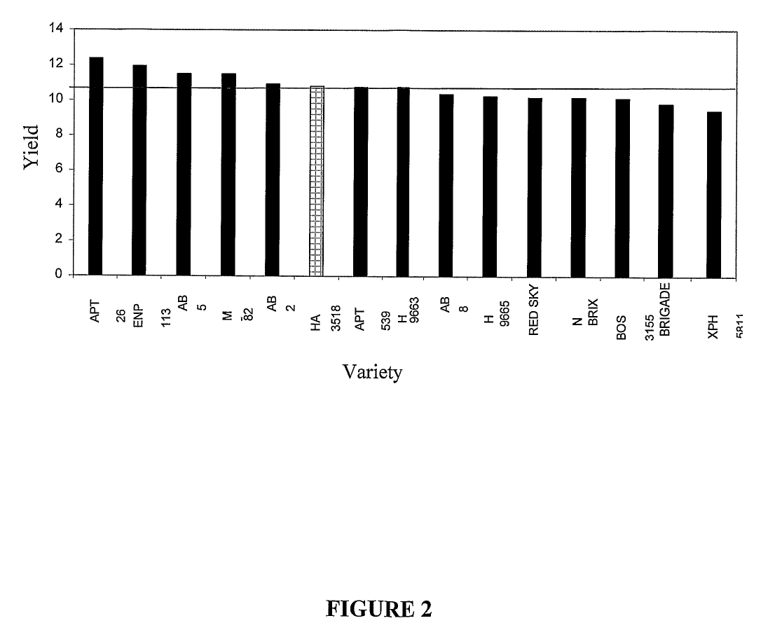

Step 2: F₂ progeny was germinated in a temperature controlled growth chamber at 26° C., 80% humidity, 90% light intensity under a yellow plastic screen, omitting transmittance of light spectra under 500 nm. This light enhances the photomorphogenetic phenotypes associate with the dg mutation, and thus seedlings which are dg homozygous could be easily selected after three days of growth (FIG. 1). The photomorphogenetic phenotypes include short and dark green stem.

Step 3: The expected dg/dg seedlings were taken for further growth in a greenhouse during the winter (Mivhor Farm, South Israel, minimum temperature 15° C.) under hydroponic conditions. Lateral shoots were pruned during the growth period to enhance the pleiotropic effects (including brittle stems, fragile leaves, shallow root system, small fruits and low crop yield). Plants were left for self-pollination and seeds (F₃) were collected from plants showing minimal pleiotropic effects. Presence of the dg mutation within the selected plants genotype was verified using specific PCR markers as exemplified herein below.

Step 4: 20 F₃ seeds from each selected plant as described in step 3 above were sown in a temperature controlled growth chamber (at 90% light intensity under yellow plastic screen, 26° C., 80% humidity). Seedlings having short nodes and dark green leaves were selected and planted in an open field during the summer and left for self-pollination until complete maturity of the fruits was obtained. Each 20-plants group was designated as specific breeding lines, and the breeding lines were subjected to further selection.

Step 5: Further selection was performed by the following sequential steps:
a) Each breeding line was examined for average performance, including: lycopene content, volume of root system, vigor, fruit size and fruit yield, leaf appearance and health and total soluble solid content (TSS/BRIX). Breeding lines showing the highest results were selected for further analysis.
b) The above described parameters were examined for individual plants within the selected breeding lines. The best plant from each group was left for self-pollination, and seeds (F₄) were collected.
c) The F₄ seeds were geminated under yellow plastic screen in a temperature controlled growth chamber at 26° C., 80% humidity, 90% light intensity. Seedlings identified by their phenotypic appearance as dg homozygous were taken for further analysis. Genomic DNA was extracted from the seedlings and the presence of the dg mutation was confirmed using dg-specific PCR markers.

The seedlings selected after step 5 (c) were dg homozygous, and therefore produced fruits comprising high lycopene content, while showing low dg-linked deleterious effects. To obtain superior commercial varieties, these F₄ plants were again crossed with a mixture of commercially valuable breeding material as described above, and steps 1 to 5 were repeated. Best performing plants (tentative parent varieties) were selected and crossed to obtain experimental F₁ hybrids. The experimental F₁ hybrids were examined during the summer in Israel, in two replications at two different locations, according to the following parameters:

Lycopene content, measured spectrophotometerically as described herein below;

Fruit crop yield measured as tons/1000 m²;

Fruit quality, scored according to the following: firmness, shape, texture (including fibrous and puffiness) and taste;

Soluble solid content (Brix), measured as described herein below;

Plant quality, scored according to the followings: vigor, plant resistance to various diseases, plant resistant to stress, foliage health and density, unity of ripening and fruit quality at ripening.

Best performing F₁ plants (i.e. plants producing fruits comprising at least 200 ppm lycopene without showing any pleiotropic effects) were identified. The tentative parent lines from which these F₁ hybrids were produced were taken for another round of selection. Best scored plants within the selected breeding lines were identified as the stable parent lines of the present invention. The parent lines have shown uniformity and stability for all traits. They were self-pollinated and planted for a sufficient number of generations, with careful attention to uniformity of plant type to ensure homozygosity and phenotypic stability. These stabilized parent plant lines were used for the production of the hybrid tomato seeds and plants of the present invention. It is to be understood that a lycopene content of 200 ppm is an average for a certain crop, and may change according to the intrinsic variation of different growth regimes, weather conditions, fruit stage of ripening etc.

According to one embodiment, the seeds and plants of the present invention are F₁ hybrid varieties designated HA3512, HA3513, HA3518 and HA3519. The F₁ hybrid varieties of the present invention are superior over the stabilized parent lines in their plant vigor and adaptation for growth in a commercial scale.

According to one currently preferred embodiment, the present invention provides tomato hybrid seeds designated HA3518. Hybrid HA3518, representative seeds of which have been deposited with the ATCC on Jan. 29, 2004 (Accession number not available) serves as an example for the hybrids of the present invention, wherein the plants grown from the seeds are homozygous for the dg mutation, produce fruit crop yield comprising an average lycopene content of at least two fold its content in currently available crop yields, and is devoid of the dig associated undesired pleiotropic traits.

According to another aspect, the present invention provides a method for producing first generation (F₁) hybrid tomato seeds.

According to one embodiment, the present invention provides a method for producing first generation hybrid seeds comprising crossing a first stable parent tomato plant with a second stable parent tomato plant and harvesting the resultant hybrid F₁ seeds, wherein the first and the second stabilized parent plants are homozygous for the dg mutation while devoid of the deleterious effects associated with the dg mutation.

According to another embodiment, the present invention also provides a first generation F₁ hybrid tomato plants that are produced by growing the hybrid tomato seeds produced by the above-described method. As exemplified herein below, the obtained F₁ hybrid plants were grown in different locations during various growth seasons. In all examined growth conditions, the high-lycopene F₁ plants according to the present invention are at least of the same quality as known commercial tomato varieties, while producing fruits having lycopene content at least two fold its content in the commercial varieties.

The present invention also relates to seeds harvested on the F₁ hybrid tomato plants and plants grown from these seeds. A common practice in plant breeding is using the method of backcrossing to develop new varieties by single trait conversion. The term single trait conversion as used herein refers to the incorporation of new single gene into a parent line wherein essentially all of the desired morphological and physiological characteristics of the parent lines are recovered in addition to the single gene transferred. The term backcrossing as used herein refers to the repeated crossing of a hybrid progeny back to one of the parental tomato plants. The parental tomato plant which contributes the gene for the desired characteristic is termed the nonrecurrent or donor parent. This terminology refers to the fact that the nonrecurrent parent is used one time in the backcross protocol and therefore does not recur. The parental tomato plant to which the gene or genes from the nonrecurrent parent are transferred is known as the recurrent parent as it is used for several rounds in the backcrossing protocol. In a typical backcross protocol, a plant from the original varieties of interest (recurrent parent) is crossed to a plant selected from second varieties (nonrecurrent parent) that carries the single gene of interest to be transferred. The resulting progeny from this cross are then crossed again to the recurrent parent and the process is repeated until a tomato plant is obtained wherein essentially all of the desired morphological and physiological characteristics of the recurrent parent are recovered in the converted plant, in addition to the single transferred gene from the nonrecurrent parent. Backcrossing methods can be used with the present invention to improve or introduce a characteristic into the parent lines

Tomato plants having within their pedigree tomato hybrid homozygous for the dg mutation producing fruit crop yield which comprises an average lycopene content of at least two fold its content in currently available crop yields, while being devoid of the undesired pleiotropic traits associated dg mutation according to the present invention are also included within the scope of the present invention.

The present invention encompasses any part of the stabilized parent plant or of the hybrid plant, including pollen, ovules and tissue cultures regenerated from these plants. Pollen and ovules are used in breeding programs, in general and as described by the present invention. Tissue culture of tomato can be used for the in vitro regeneration of a tomato plant as is well known in the art.

According to another aspect, the present invention provides high lycopene tomato fruit. The fruits can be marketed as a fresh product or can serve as a source for processed high lycopene tomato products and for purified lycopene. Tomato and tomato product consumption is growing constantly due to the development of new tomato varieties which permit supply all year long, and to the increased awareness to the general nutritional benefit of fruits. Epidemiological studies showing the beneficial effects of frequent and regular consumption of tomatoes or tomato products in reducing the risk of chronic disorders, including cancer and cardiac and circulatory disorders have further increased the demand for tomatoes, particularly for tomato fruit comprising higher levels of lycopene. The requirements for purified lycopene as a natural colorant and moreover as nutritional supplement have also increased dramatically during the last decade. Thus, the high lycopene fruits produced by the plants of the present invention are particularly suitable to meet the above-described demands.

According to one embodiment, the average lycopene content of the fruits is at least two fold it content compared to its content in other commercially available fruits. According to one currently preferred embodiment, the average lycopene content of the fruits is at least 200 ppm.

The novel aspect of the present invention is in providing plants that produce fruit in a commercial scale, wherein the average lycopene content within the fruit crop is at least two fold the lycopene content found in fruit crops produced by other commercial varieties. Tomato plants homozygous for the dg mutation were previously disclosed. In order to reach maturity and produce fruits containing high lycopene amounts such plants had to be grown under unique and favorable conditions, typically in controlled green houses or growth chambers, and only few fruits could be obtained on each plant. Attempts were also made to incorporate the dg mutation to commercially valuable varieties; however, to the best of our knowledge, the present invention is the first to provide commercial tomato varieties, having at least comparable horticultural performance as other commercial varieties, while producing high amounts of lycopene, typically an average of at least 200 ppm plus or minus the standard error from the mean.

The principle of the invention may be better understood with reference to the following non-limiting examples.

### EXAMPLES

Measurements of Lycopene Content

Lycopene content can be measured by various techniques as is known to a person skilled in the art. Typically, lycopene was extracted from pericarp tissue of fresh ripe-red fruits. Fruits were sampled as to represent the total yield. Pericarp tissue samples were minced to puree in a blender. Lycopene was extracted with extraction buffer consisted of n-hexane:isopropanol:acetone (2:1:1). Phase separation was achieved by the addition of sufficient amounts of NaCl 0.1M. The organic upper phase was collected for analysis. Lycopene concentration was calculated according to its absorbance at 472 nm using E1% of 3,450.

Measurements of Total Soluble Solid Concentration (BRIX)

Soluble solid concentration was measured in the juice of the ripe tomato. Few drops were placed on a refractometer (TAMCO, Japan), and the BRIX values were read.

### Example 1

### Production of Hybrid HA3518

Hybrid HA3518, seeds of which have been deposited with the ATCC on Jan. 29, 2004 serve as an example for the teaching of the present invention.

Seedlings obtained according to steps 1 to 5 described herein above served as a source for the production of the HA3518 hybrid. Lines stabilized for the dg mutation were selected as parent lines The presence of the dg mutation was verified by PCR reaction using a dg-probe, and by the phenotypic effects of the mutation. This parent plants showed normal growth pattern with a developed roots system and healthy leaves, and produced fruit containing lycopene content over 200 ppm. 300 F₁ hybrids were produced using these lines and other commercial valuable material. The F₁ hybrids were planted (in two replicates for each hybrid) during the summer of 2001 in two different locations in Israel: Mivhor farm (South Israel) and Ramat-David (North Israel). Plants were left for self-pollination until maturity, and fruit lycopene content was measured. Eight hybrids were found to contain high lycopene content (over 200 ppm), and normal growth pattern. The parent lines of these 8 selected hybrids were self pollinated and planted for sufficient number of generations to obtain stabilized parent plant, homozygous for the dg mutation while devoid of: poor germination rate; shallow root system; brittle stems; thin and/or fragile leaves; small fruit; low crop yield. The eight hybrids were then produced again using the stable parent lines, hybrid HA3518 serving as a representative example. The hybrids were planted in ten different locations in Israel, at different planting dates during the year of 2002. Seedlings were planted mechanically; growth regime was as common to commercial varieties at the different locations; and fruit harvest was also performed mechanically as is known for commercial varieties. Fruit crop yield was measured as Kg/m². Average crop yield for hybrid HA3518 was at least 9 Kg/m² with an average lycopene content of 204 ppm. This average lycopene content was obtained from yield harvested in all the location examined, therefore included also plots in which growth was interrupted due to local unfavorable growth conditions and stress. Highest lycopene content measured was 270 ppm and highest yield obtained was 12.7 Kg/m². The stable parent from which this HA3518 hybrid was produced were further self-pollinated and selected for best-performing plants in terms of horticulture measures. The new stabilized parents were used to produce a second generation of hybrid HA3518, representative seeds of which have been deposited with the ATCC on Jan. 29, 2004.

The HA3518 hybrid was planted during the year 2003 in 13 different locations as described in table 1 below covering about 430 1000 m². Ripen fruits were harvested, yield was weighed and average lycopene content was measured, as described in table 1. This large-scale trial represents various growth conditions, including local whether hazards and sub-optimal growth regime. Nevertheless, the average yield obtained from entire plot examined was commercially acceptable, and the average lycopene content of 235 ppm is significantly high. FIG. 1 shows comparison of fruit yield of various commercial varieties and hybrid HA3518 obtained at one location (Akko, North Israel). The average crop yield of hybrid HA3518 was 10.8 Kg/m², which is considered as average to high yield for a commercial variety.

### Example 2

### Lycopene Content of Novel Hybrids Compared to Commercial Varieties

Common commercial varieties “Brigade” and “HA 3303”, and the new varieties of the present invention, Hybrids HA3512, HA3513 and HA3518 were planted in Ramat Hagolan, Israel. The plants were grown under ordinary growth regime, as is known for the commercial varieties until fruit ripening. Each variety was planted in at least four replicates. Fruit were harvested on August 2002. The following parameters were measured: BRIX; pH; lycopene concentration; and average weight of the sample. Table 2 (raw data) and Table 3 (summary) below summarize the results.

TABLE 2
lycopene content in novel vs. common tomato varieties-raw data

| | | | Average sample | | Lycopene | | |
|---|---|---|---|---|---|---|---|
| | | | weight | Abs | Concentration | | |
| Serial No. | Plot | Varieties | (gr) | 472 nm | ppm | pH | BRIX |
| 626 | 19 | Brigade | 10.378 | 0.322 | 90 | 4.51 | 3.7 |
| 627 | 19 | Brigade | 10.460 | 0.305 | 85 | 4.43 | 4.3 |
| 631 | 22 | HA 3512 | 10.404 | 0.929 | 259 | 4.90 | 4.3 |
| 632 | 22 | HA 3512 | 10.480 | 0.783 | 217 | 4.89 | 5.2 |
| 633 | 22 | HA 3512 | 11.515 | 1.006 | 253 | 4.99 | 4.8 |
| 649 | 34 | Brigade | 10.567 | 0.313 | 86 | 4.63 | 3.7 |
| 650 | 34 | Brigade | 10.110 | 0.305 | 87 | 4.66 | 3.9 |
| 659 | 43 | HA 3518 | 10.711 | 0.765 | 207 | 4.75 | 5.2 |
| 660 | 43 | HA 3518 | 10.671 | 0.796 | 216 | 4.69 | 4.4 |
| 661 | 43 | HA 3518 | 10.198 | 0.779 | 221 | 4.75 | 4.2 |
| 665 | 47 | HA 3512 | 11.106 | 0.918 | 240 | 4.73 | 4.6 |
| 666 | 47 | HA 3512 | 10.427 | 0.865 | 240 | 4.85 | 4.1 |
| 667 | 47 | HA 3512 | 11.653 | 0.890 | 221 | 4.74 | 4.5 |
| 668 | 52 | HA 3518 | 10.249 | 0.811 | 229 | 4.76 | 4.1 |
| 669 | 52 | HA 3518 | 10.281 | 0.820 | 231 | 4.81 | 4.2 |
| 670 | 52 | HA 3518 | 10.300 | 0.942 | 265 | 4.83 | 4.8 |
| 671 | 54 | HA 3512 | 10.827 | 0.933 | 250 | 4.85 | 4.5 |
| 672 | 54 | HA 3512 | 10.711 | 0.905 | 245 | 4.86 | 4.2 |
| 673 | 54 | HA 3512 | 9.296 | 0.854 | 266 | 4.72 | 4.6 |
| 674 | 55 | HA 3518 | 10.080 | 0.759 | 218 | 4.79 | 4.6 |
| 675 | 55 | HA 3518 | 10.094 | 0.736 | 211 | 4.63 | 4.8 |
| 676 | 55 | HA 3518 | 10.143 | 0.793 | 227 | 4.72 | 4.3 |
| 677 | 57 | HA 3303 | 9.868 | 0.301 | 88 | 4.68 | 3.6 |
| 678 | 58 | HA 3303 | 10.635 | 0.339 | 92 | 4.64 | 3.4 |
| 679 | 44 | HA 3513 | 10.161 | 0.732 | 209 | 4.68 | 4.7 |
| 680 | 44 | HA 3513 | 10.173 | 0.724 | 206 | 4.80 | 4.3 |
| 681 | 44 | HA 3513 | 10.495 | 0.761 | 210 | 4.67 | 4.8 |
| 682 | 37 | HA 3513 | 10.386 | 0.687 | 192 | 4.75 | 4.3 |
| 683 | 37 | HA 3513 | 10.014 | 0.692 | 200 | 4.71 | 4.9 |
| 684 | 37 | HA 3513 | 9.941 | 0.648 | 189 | 4.72 | 4.1 |

TABLE 3
lycopene content in novel vs. common tomato varieties-average

| | Lycopene | | |
|---|---|---|---|
| | Concentration | | |
| Varieties | ppm | pH | BRIX |
| Brigade | 87 | 4.6 | 3.9 |
| (Control) | | | |
| 3303 | 90 | 4.66 | 3.5 |
| (Control) | | | |
| 3512 | 243 | 4.84 | 4.5 |
| 3513 | 201 | 4.72 | 4.5 |
| 3518 | 225 | 4.75 | 4.5 |

Table 2 above clearly shows that the new high lycopene hybrids of the present invention are superior over the common commercial varieties examined, as they produce fruits with total soluble solids and lycopene content significantly higher compared to the commercial varieties. The lycopene content according to this example is 2.5 fold higher.

### Example 3

### Genotypic Identification of the dg Mutation

The PCR primers used to amplify the tomato DET1 genomic DNA fragment flanking the dg mutation locus were: 5′-TTC TTC GGA TTG TCC ATG GT-3′ AND 5′CAC CAA TGC TAT GTG CCA AA-3′.

The amplification reactions (25 μl final volume) were performed with 10 ng of template DNA, 25 mM of TAPS (pH=9.3 at 25° C.), 50 mM of KCl, 2 mM of MgCl₂, 1 mM of B-mercaptoethanol, 0.2 mM of each of the four deoxyribonucleotide triphosphates (dATP, dCTP, dGTP and dTTP), 10 ng of each of two primers and 1 unit of thermostable Taq DNA polymerase (SuperNova Taq polymerase, Madi Ltd., Rishon Le Zion, Israel). Reactions were carried out in an automated thermocycler (MJ Research Inc., Watertown, Mass., USA). Initial incubation was at 94° C. for 1 min, followed by 34 cycles of denaturation at 94° C. for 1 min, annealing at 55° C. for 1 min and polymerization at 72° C. was carried out, for 3 min, after the above cycles have been completed. The PCR amplification products were visualized by electrophoresis in 1.00% agarose gels and detected by staining with ethidium bromide.

1. A robust tomato variety homozygous for the dg mutation, wherein tomatoes grown from this variety have an average lycopene content at least two fold its average content in currently available varieties, while being devoid of deleterious traits associated with the dg mutation, when measured at peak lycopene content.

2. The variety of claim 1, wherein the variety is a stable parent line.

3. The variety of claim 1, wherein the variety is a hybrid.

4. The variety of claim 1 wherein the average lycopene content in the fruit crop is at least 200 ppm plus or minus the standard error from the mean.

5. The variety of claim 1 which is devoid of dg-linked deleterious traits selected from the group consisting of poor germination rate, shallow root system, brittle stems, thin and/or fragile leaves, premature defoliation, low yield, and small fruit.

6. The variety of claim 3 selected from the group consisting of hybrids designated HA3512, HA3513, HA3518 and HA3519.

7. The variety of claim 6 designated HA3518, a sample of the seed of this variety having been deposited with the American Type Culture Collection under designation No. PTA-5796.

8. A seed of a robust tomato variety homozygous for the dg mutation, wherein tomatoes grown from this variety have an average lycopene content at least two fold its average content in currently available varieties, while being devoid of deleterious traits associated with the dg mutation, when measured at peak lycopene content.

9. The seed of claim 8, wherein the variety is a stable parent line.

10. The seed of claim 8 wherein the variety is a hybrid.

11. The seed of claim 8 wherein the average lycopene content in the fruit crop is at least 200 ppm plus or minus the standard error of the mean.

12. The seed of claim 8 which is devoid of dg-linked deleterious traits selected from the group consisting of poor germination rate, shallow root system, brittle stems, thin and/or fragile leaves, premature defoliation, low yield and small fruit.

13. The seed of claim 10 selected from the group consisting of hybrids designated HA3512, HA3513, HA3518 and HA3519.

14. The seed of claim 13 designated HA3518, a sample of the seed of this variety having been deposited with the American Type Culture Collection under designation No. PTA-5796.

15. A tomato plant, or part thereof, produced by growing the seed of claim 8.

16. Pollen of the plant of claim 15.

17. An ovule of the plant of claim 15.

18. The plant of claim 15 further comprising an additional trait consisting of herbicide resistance, insect resistance, resistance to bacterial, fungal or viral disease, male sterility and improved nutritional value.

19. The plant of claim 15 further comprising an additional trait selected from at least one type of disease resistance and at least one type of stress resistance.

20. The plant of claim 15 further comprising an additional trait introduced by genetic transformation.

21. The plant of claim 18 further comprising an additional trait introduced by genetic transformation.

22. The tomato plant, or part thereof, of claim 15, wherein the plant or parts thereof have been transformed so that its genomic material contains one or more transgenes operably linked to one or more regulatory elements.

23. The tomato plant, or part thereof, of claim 18, wherein the plant or parts thereof have been transformed so that its genomic material contains one or more transgenes operably linked to one or more regulatory elements.

24. A tissue culture of regenerable cells of a tomato plant of claim 15.

25. A tissue culture according to claim 24, comprising cells or protoplasts from a tissue selected from the group consisting of leaves, pollen, embryos, roots, root tips, anthers, flowers, fruit and seeds.

26. The tissue culture of regenerable cells of claim 24, wherein the tissue regenerates plants capable of expressing all the morphological and physiological characteristics of the hybrid HA3518, a sample of a seed of said variety having been deposited with the American Type Culture Collection under designation No. PTA-5796.

27. A tomato plant regenerated from the tissue culture of claim 24, capable of expressing all the morphological and physiological characteristics of the hybrid HA3518, a sample of a seed of said variety having been deposited with the American Type Culture Collection under designation No. PTA-5796.

28. A method for producing a hybrid tomato seed comprising crossing a first parent tomato plant with a second parent tomato plant and harvesting the resultant hybrid F₁ seed, wherein at least one of the first or the second parent tomato plant is a variety according to claim 2.

29. A hybrid tomato seed produced by the method of claim 28.

30. A hybrid tomato plant, or parts thereof, produced by growing the hybrid tomato seed of claim 29.

31. Tomato seed produced by growing the hybrid tomato plant of claim 30.

32. A method of producing a tomato plant derived from a hybrid tomato variety according to claim 3, comprising:
a. crossing a first plant that is a hybrid plant homozygous for the dg mutation according to the present invention with a second tomato plant to yield first progeny seeds;
b. growing the first progeny seed under suitable plant growth conditions to yield an F₁ tomato plant of the first hybrid plant; optionally
c. crossing the plant obtained in step (b) with itself or with a third tomato plant to yield second progeny seeds derived from said first hybrid plant;
d. growing the second progeny seed under suitable plant growth conditions to yield additional tomato plant derived of said first hybrid plant; and further optionally
e. repeating the steps of crossing and growing from 1 to 5 or more times to generate further tomato plants derived from said first hybrid plant.

33. The method of claim 32 wherein the hybrid variety is selected from the hybrids designated HA3512, HA3513, HA3518 and HA3519.

34. The method of claim 33, wherein the hybrid variety is HA3518, a sample of a seed of said variety having been deposited with the American Type Culture Collection under designation No. PTA-5796.

35. A method for producing a tomato plant that contains in its genetic material at least one transgene, comprising crossing the tomato plant of claim 20 with either a second plant of another tomato variety or a non-transformed tomato plant according to claim 1, so that the genetic material of the progeny that results from the cross contains the at least one transgene operably linked to a regulatory element.

36. A tomato plant, or part thereof, produced by the method of claim 35.

37. A tomato plant according to claim 15 further comprising a single trait conversion.

38. The tomato plant of claim 37, wherein the single trait confers a characteristic selected from the group consisting of herbicide resistance, insect resistance, resistance to bacterial, fungal or viral disease, male sterility and improved nutritional value.
